(19) United States
(12) Patent Application Publication
(10) Pub. No.: US 2004/0234026 A1
(43) Pub. Date: Nov. 25, 2004

(54) Mammographic image obtaining apparatus

(75) Inventor: Takashi Shoji, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/841,539

(22) Filed: May 10, 2004

(30) Foreign Application Priority Data
May 23, 2003 (JP) 146213/2003

(51) Int. Cl. A61B 6/04

(52) U.S. Cl. 378/37

(57) ABSTRACT

A mammographic image obtaining apparatus having an automatic pressing plate withdrawing mechanism in which S/N ratio for the image signals is prevented from degrading. After a mammographic radiation image is recorded on the solid-state detector by fixing the breast on the photography platform with the pressing plate, the voltage applied to the solid-state detector is terminated, and the pressing plate moving means is activated to withdraw the pressing plate from a position to press the breast to a position to release the pressing based on the instruction of the control means before the image signals are read out from the solid-state detector. Thereafter, the image signal reading is performed.

### BACKGROUND OF THE INVENTION

1. Field of the Invention

The present invention relates to a mammographic image obtaining system for obtaining a mammographic radiation image using a solid-state detector.

2. Description of the Related Art

In medical X-ray and other radiography, a system for reading out radiation image information using a solid-state detector has been known as described, for example, in U.S. Pat. No. 6,268,614 and Japanese Unexamined Patent Publication No. 2000-284056, etc. The system uses a photoconductor made of, for example, an a-Se(amorphous Selenium) plate which is sensitive to radiation including X-ray, as a solid-state detector in order to reduce the dose of radiation exposed to a subject and improve diagnostic capabilities, and reads out an electrostatic latent image, i.e., radiation image information by exposing the solid-state detector to recording radiation (recording light), such as an X-ray carrying radiation image information to store the charges of the latent image representing the radiation image information into the storage section of the solid-state detector, and thereafter scanning the solid-state detector with a reading electromagnetic wave (reading light) such as a laser beam and detecting electric currents generated in the solid-state detector through flat or striped electrodes provided on both sides of the detector.

Currently, a mammographic image obtaining system for obtaining a mammographic radiation image using the solid-state detector described above has been contemplated.

The mammographic image obtaining system described above obtains a mammographic radiation image by pressing the breast of a subject onto a photography platform having a solid-state detector therein using a pressing plate and directing radiation toward the breast from above. It is very painful for the subject that its breast is being pressed by the pressing plate onto the photography platform, so that in some mammographic image obtaining systems, the pressing plate is automatically withdrawn as soon as the imaging is completed in order to alleviate the pain of the subject.

The mammographic image obtaining system reads out the electrostatic latent image or image signals from the solid-state detector after imaging. In the system in which the pressing plate is withdrawn automatically as soon as imaging is completed, the image signal reading and withdrawal of the pressing plate may occur simultaneously.

If the pressing plate is moved during the image signal reading, the connecting means (e.g., flexible board) connecting the solid-state detector and printed-circuit board may vibrate due to vibrations arising from the movement of the pressing plate, and the noise generated by the vibration may be added to the image signals, causing degradation in S/N ratio for the image signals.

Further, the capacitive coupling may occur between each of the conductive layers of the detector and the X-ray entrance face of the housing due large dimensions (e.g., 18 cm×18 cm) of the detector, and the capacitance of the coupling may vary with time, which may also cause the addition of noise to the image signals, causing degradation in S/N ratio for the image signals.

Still further, an electric motor is generally used for moving the pressing plate, and if the motor is activated during the image signal reading, then noise may be generated in the power supply line that may cause degradation in S/N ratio for the image signals.

### SUMMARY OF THE INVENTION

The present invention has been made in recognition of the circumstance described above, and it is an object of the present invention to provide a mammographic image obtaining apparatus having a pressing plate with drawing mechanism in which S/N ratio for the image signals is effectively prevented from degrading.

The mammographic image obtaining apparatus according to the present invention comprises a solid-state detector that records image information when exposed to radiation carrying the image information and outputs image signals representing the recorded image information; a reading means for reading out the image signals outputted from the solid-state detector; a photography platform having the solid-state detector; a pressing plate for pressing the breast of a subject onto the photography platform; a moving means for moving the pressing plate between a position to press the breast of the subject and a position to release the pressing; and a control means for controlling the moving means, wherein the control means controls the moving means such that the pressing plate is moved by the moving means while the image signal reading is not being performed by the reading means.

The term “solid-state detector” as used herein means a detector that detects the radiation carrying image information of a subject and outputs image signals representing the radiation image information of the subject, in which the radiation entered into the detector is converted to electric charges directly or after converted to light in order to be stored into the storage section of the detector, and thereafter the image signals representing the radiation image of the subject is obtained by outputting the electric charges stored in the storage section of the detector to outside.

Various types of solid-state detectors are available. For example, from the aspect of charge generating process in which radiation is converted to electric charges, the detectors are categorized into the optical conversion system in which signal charges obtained by detecting the fluorescence emitted from a fluorescent material when exposed to radiation with an optoelectronic conversion device are stored into the storage section of the device before being converted to image signals(electrical signals) and outputted therefrom, the direct conversion system in which electric charges generated by a conductive material when exposed to radiation are collected by charge collecting electrodes and stored into the storage section of the detector before being converted to electrical signals and outputted therefrom, or the like, and from the aspect of the charge reading out process of the detector in which the stored electric charges are read out from the detector to outside, they are categorized into the TFT (thin film transistor) reading system in which the charges are read out by scan-driving the TFT connected to the storage section, the optical reading system in which the charges are read out by exposing the detector to reading light (a reading electromagnetic wave), or the like. In addition, a solid-state detector of improved direct conversion system which is a combined system of the direct conversion system and optical reading system described above, which has been proposed by the applicant as described in U.S. Pat. No. 6,268,614 and Japanese Unexamined Patent Publication No. 2000-284056, is also available. The mammographic image obtaining apparatus according to the present invention may use any type of solid-state detector described above.

The term “controls the moving means such that the pressing plate is moved by the moving means while the image signal reading is not being performed by the reading means” means that the control means controls the moving means such that the pressing plate is moved by the moving means after the image signal reading is completed or the image signal reading is performed after the pressing plate is moved for the apparatus that releases the pressing plate automatically after imagining, or such that the movement of the pressing plate is inhibited during the image signal reading for the apparatus in which the pressing plate is released manually after imaging.

The present invention may use a solid-state detector comprising a set of layers layered in the order of a first conductive layer, at least one photoconductive layer, and a second conductive layer, and records image information as an electrostatic latent image when exposed to radiation carrying the image information while a predetermined voltage is being applied between the first and second conductive layers, and generates currents as image signals when scanned with reading light after the first and second conductive layers are short-circuited.

When the aforementioned solid-state detector is used, it is preferable that the control means controls the moving means such that the pressing plate is moved by the moving means while the image signal reading is not being performed by the reading means and voltage is not being applied between the first and second conductive layers.

The mammographic image obtaining apparatus according to the present invention comprises a photography platform having a solid-state detector; a pressing plate for pressing the breast of a subject onto the photography platform; a moving means for moving the pressing plate between a position to press the breast of the subject and a position to release the pressing; a control means for controlling the moving means; and a reading means for reading out the image signals outputted from the solid-state detector, wherein the control means controls the moving means such that the pressing plate is moved while the image signal reading is not being performed by the reading means, so that the noise due to the movement of the pressing plate is prevented from generating during the image signal reading, thereby degradation in S/N ratio for the image signals may be avoided.

### BRIEF DESCRIPTION OF THE DRAWINGS

FIG. 1 is a schematic diagram of the mammographic image obtaining apparatus according to a preferred embodiment of the present invention.

FIG. 2 a schematic diagram of a photography platform of the mammographic image obtaining apparatus shown in FIG. 1 illustrating the inside thereof.

FIG. 3 is a schematic diagram of a solid-state detector used in the mammographic image obtaining apparatus shown in FIG. 1.

FIG. 4 is a block diagram illustrating the details of the current detecting means and high-voltage power supply section of the mammographic image obtaining apparatus shown in FIG. 1, and the connections between these and the solid-state detector.

FIG. 5A is a timing chart illustrating each of the operational timings from imaging to reading in the mammographic image obtaining apparatus shown in FIG. 1.

FIG. 5B is a timing chart illustrating each of the operational timings from imaging to reading in the mammographic image obtaining apparatus shown in FIG. 1.

### DESCRIPTION OF THE PREFERRED EMBODIMENTS

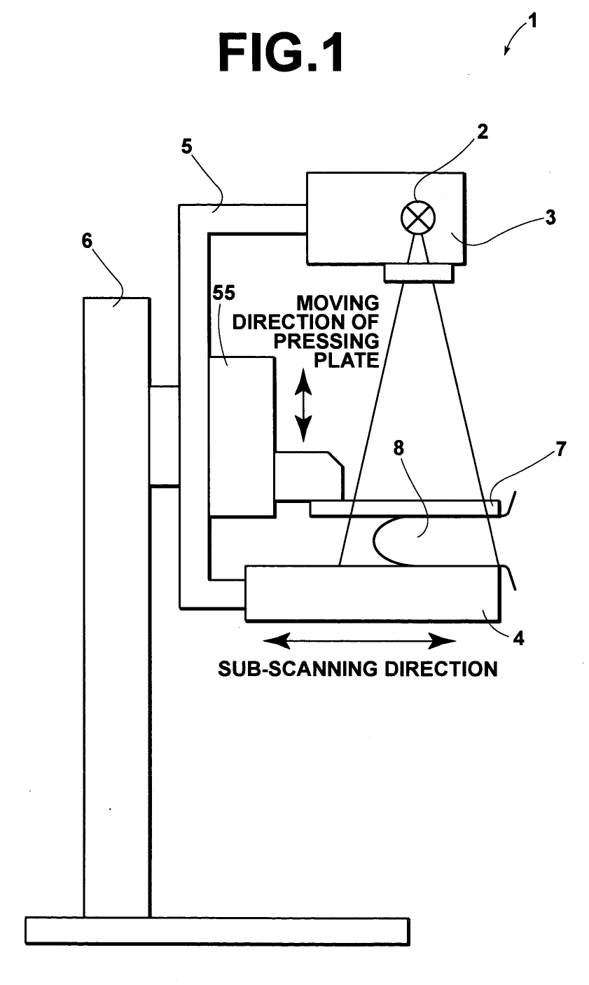

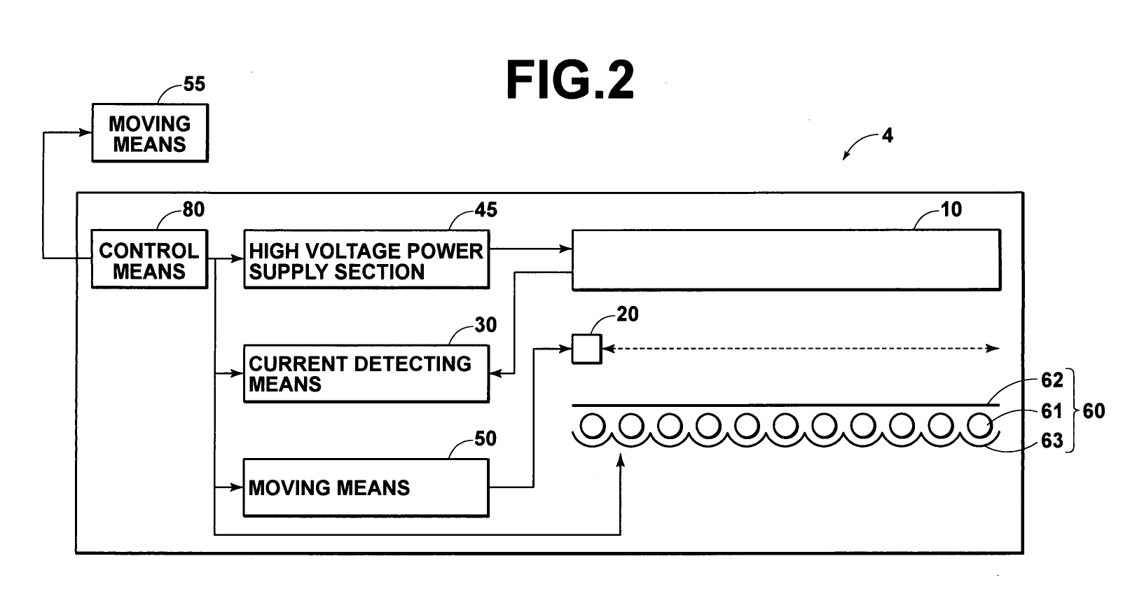

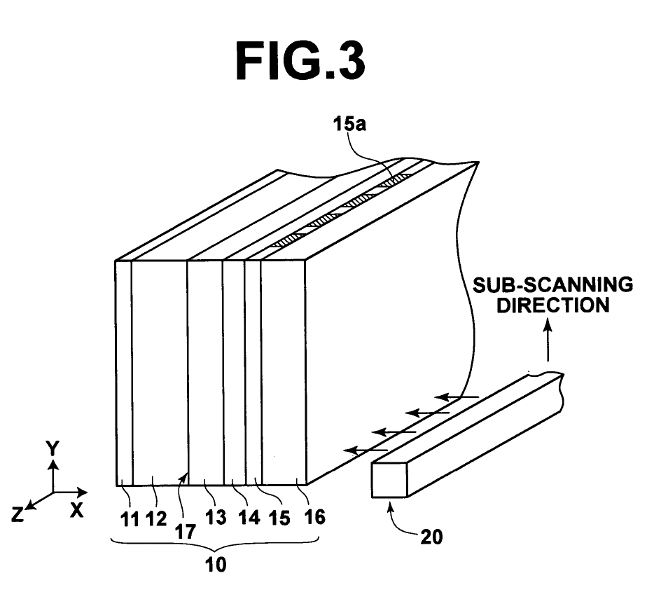

Hereinafter, the preferred embodiment of the present invention will be described in detail with reference to the accompanying drawings. FIG. 1 is a schematic diagram of the mammographic image obtaining apparatus according to a preferred embodiment of the present invention. FIG. 2 is a schematic diagram of a photography platform of the apparatus shown in FIG. 1, illustrating the inside thereof, and FIG. 3 is a schematic diagram of a solid-state detector used for the apparatus shown in FIG. 1.

The mammographic image obtaining apparatus 1 shown in FIG. 1 comprises a radiation source storage section 3 having a radiation source 2 therein; a photography platform comprising a housing 4 having a solid-state detector 10 and others therein; an arm 5 for connecting the radiation source storage section 3 and the photography platform in face to face; and a base 6 for fixing the arm 5.

Further, the arm 5 has a pressing plate 7 for pressing a breast 8 of a subject onto the housing 4 from above and holding it in place, and a pressing plate moving means 55 for automatically moving the pressing plate 7 based on the instruction from a control means 80 to be described hereinafter. The pressing plate moving means 55 comprises a linear motor which is not shown, and translates the pressing plate 7 back and forth between a position to press the breast 8 onto the housing 4 and a position to release the pressing.

The housing 4 includes therein, the solid-state detector 10 which is the imaging device of the apparatus; a reading light source section 20 used for reading out radiation image information recorded on the solid-state detector 10; a reading light source section moving means 50 for moving the reading light source section 20 in the sub-scanning direction; a current detecting means 30 for obtaining image signals by detecting currents flowing out from the solid-state detector 10 when scanned with the reading light source section 20; a high-voltage power supply section 45 for applying a predetermined voltage to the solid-state detector 10; a pre-exposure light source section 60 for applying pre-exposure light on the solid-sate detector 10 prior to imaging; and a control means 80 for controlling the reading light source section 20, current detecting means 30, high-voltage power supply section 45, pre-exposure light source section 60, and moving means 50 and 55.

The solid-state detector records radiation image information as an electrostatic latent image, and generates currents in accordance with the electrostatic latent image when scanned with the reading light. More specifically, as illustrated in FIG. 3, it is formed on a glass substrate 16 and comprises a set of layers layered in the order of a first conductive layer 11 having transparency to radiation (hereinafter referred to as “recording light”) transmitted through the breast 8, a recording photoconductive layer 12 that takes on conductivity by generating electric charges when exposed to the recording light, a charge transport layer 13 that acts as substantially an insulator against the charges having the same polarity as that of the charges of the latent image charged on the first conductive layer 11 and as substantially a conductor for charges having the transport polarity, which is the opposite polarity to that of the charges of the latent image, a reading photoconductive layer 14 that takes on conductivity by generating electric charges when exposed to the reading light, and a second conductive layer 15 having transparency to the reading light. A storage section 17 is formed at the interface between the recording photoconductive layer 12 and the charge transport layer 13.

Each of the first conductive layers 11 and second conductive layer 15 serves as an electrode. The electrode of the first conductive layer 11 is a two-dimensional flat electrode, and the electrode of the second conductive layer 15 is a striped electrode comprising a number of elements 15a (wire electrodes) disposed in stripes at a pixel pitch as shown in hatched lines in the FIG. 3 (refer to, for example, in U.S. Pat No. 6,268,614). The arranging direction of the elements 15a corresponds to the main scanning direction, and the longitudinal direction of the elements 15a corresponds to the sub-scanning direction.

The reading light source section 20 comprises a linear light source composed of a plurality of LED chips disposed linearly, and an optical system for irradiating the light emitted from the linear light source onto the solid-state detector 10 as linear light. The solid-state detector 10 is entirely exposed to the reading light by scanning the reading light source section 20 with the moving means 50 comprised of a linear motor over the solid-state detector 10, with a necessary distance therebetween being maintained, in the longitudinal direction of the striped electrodes 15a of the solid-state detector 10, i.e., the sub-scanning direction. The reading light scanning means is formed of the reading light source section 20 and moving means 50.

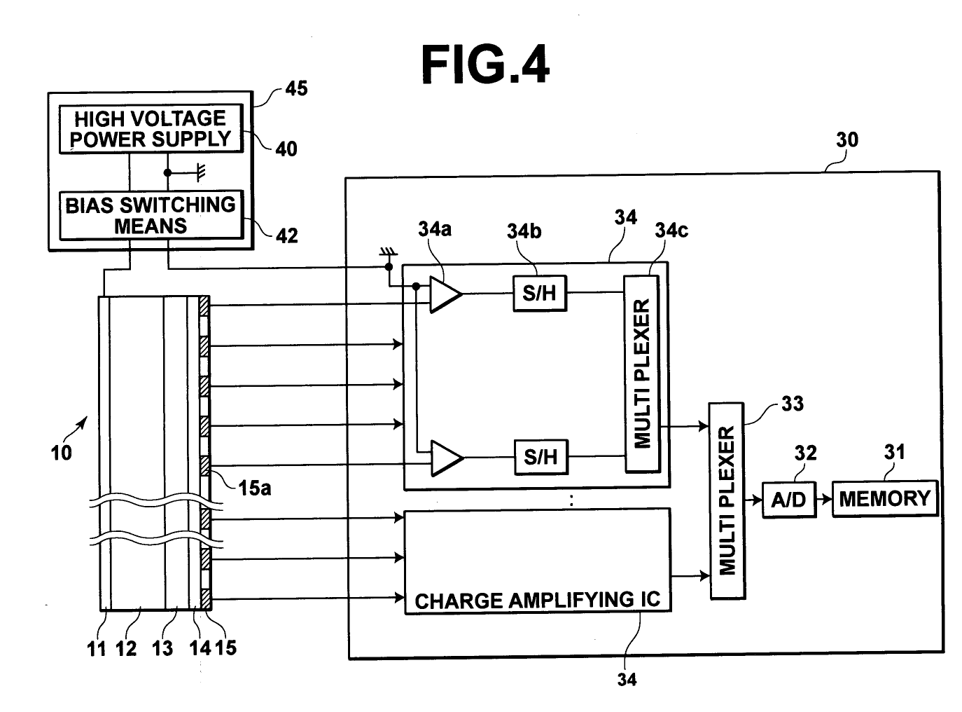

FIG. 4 is a block diagram illustrating the details of the current detecting means 30 and high-voltage power supply section 45 provided inside the housing 4 of the photography platform and connections between these and the solid-state detector 10.

The high-voltage power supply section 45 comprises a combined circuitry of a high-voltage power supply 40 and bias switching means 42. The high-voltage power supply 40 is connected to the solid-state detector 10 through the bias switching means 42 for providing biasing/short-circuiting the solid-state detector 10. The circuit described above is designed to prevent over-currents from flowing arising from charging and discharging by limiting the peak current at the time of switching, in order to protect the portions of the apparatus where a large current flows.

The current detecting means 30 comprises a memory 31; an A/D conversion section 32; a multiplexer 33; and a charge amplifying IC 34. In the preferred embodiment of the present invention, several to dozens of charge amplifying ICs 34 are provided in total, and each group of adjoining several elements 15a is connected sequentially to each of the charge amplifying ICs 34, instead of connecting all the elements 15a to a single charge amplifying IC 34.

Each of the charge amplifying ICs 34 has a number of charge amplifiers 34a, each connected to each of the elements 15a of the solid-state detector 10 respectively, and sample-and-hold (S/H) circuits 34b, each connected to each of the charge amplifiers 34a respectively, and a multiplexer 34c for multiplexing the signals from respective sample-and-hold circuits 34b. The currents flowing out from the solid-state detector 10 are converted to voltages by respective charge amplifiers 34a, which are then sampled at a predetermined timing and held by the respective sample-and-hold circuits. Then, the voltages, each corresponding to each of the elements 15a, held by the respective sample-and-hold circuits are sequentially outputted from the multiplexer 34c such that they are switched sequentially in the order in which the corresponding group of elements 15a is arranged (which constitutes a part of the main scanning). The voltages sequentially outputted from the multiplexer 34c are inputted to the multiplexer 33, and the voltages, each corresponding to each of the elements 15a, are sequentially outputted from the multiplexer 33 such that they are sequentially switched in the order in which the elements 15a are arranged to complete the main scanning. The signals sequentially outputted from the multiplexer 33 are converted to digital signals through the A/D conversion section 32, and the digitized signals are stored into the memory 31.

The pre-exposure light source section 60 requires a light source capable of emitting/quenching light immediately with an extremely short-lived afterglow. In the preferred embodiment of the present invention, a rare gas fluorescent lamp with external electrodes is used. More specifically, as shown in FIG. 2, the pre-exposure light source section 60 comprises a plurality of rare gas fluorescent lamps with external electrodes 61 extending backward from the surface of the FIG. 2; a wavelength selection filter 62 inserted between the fluorescent lamps 61 and the solid-state detector 10; and a light reflector 63 placed on the back of the fluorescent lamps to effectively reflect the light emitted from the fluorescent lamps 61 onto the solid-state detector 10. The pre-exposure light is required to be applied only over the second electrode layer 15 of the solid-state detector 10, and no particular light converging means is required. However, a narrower distribution of illuminance is preferable. As for the light source, for example, LED chips disposed in a two-dimensional plane may be used instead of the fluorescent lamps.

In reading out an electrostatic latent image from the solid-state detector, all of the charges representing the latent image may be read out in principle. But, in some cases, not all of the latent image may be read out and some of them remain in the solid-state detector 10 as the residual charges. In addition, in recording an electrostatic latent image on the solid-state detector 10, a high voltage is applied to the solid-state detector 10 before being exposed to the recording light, which may generate a dark current, thereby charges due to the dark current (dark current charges) may also be accumulated in the solid-state detector 10. Further, it is known that various kinds of charges arising from causes other that those described above are accumulated in the solid-state detector before being exposed to the recording light. These unwanted charges, including the residual and dark current charges accumulated in the solid-state detector 10 before being exposed to the recording light are added to the charges carrying image information stored in the solid-state detector 10 when exposed to the recording light, thus the output signals contain in effect the unwanted signal components due to the unwanted charges other than those based on the charges carrying image information when the electrostatic latent image is read out from the solid-state detector 10, causing problems such as image lag, degraded signal-to-noise ratio, and the like.

The purpose of the pre-exposure is to eliminate these problems, including the image lag and degraded S/N ratio by erasing the unwanted charges accumulated in the solid-state detector 10 before being exposed to the recording light.

Hereinafter, the operation of the mammographic image obtaining apparatus 1 configured in the aforementioned manner will be described.

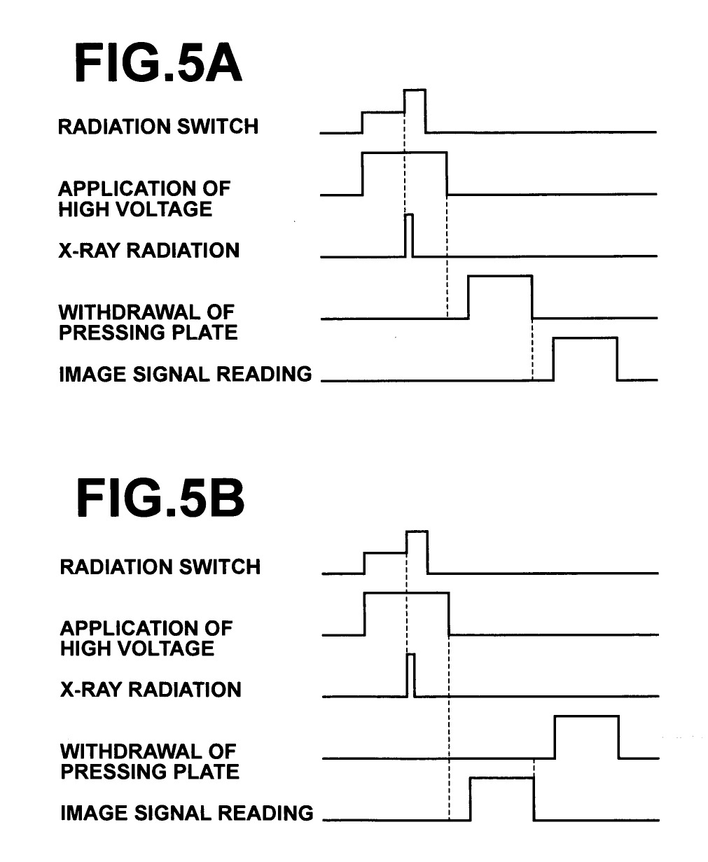

FIG. 5A is a timing chart illustrating respective operational timings from imaging to reading in the apparatus 1.

At the time of imaging, the solid-state detector 10 is first exposed to the pre-exposure light before imaging to erase the unwanted charges accumulated therein. Pre-exposure process may be implemented before or after the application of voltage to the solid-state detector 10. Further it may be such that the pre-exposure light is switched on before the application of the voltage to the detector 10 and switched off after the voltage is applied thereto. Based on the manual instruction from the imaging staff, the control means 80 drives the pressing plate moving means 55 to move the pressing plate 7 to the position to press the breast 8, thereby the breast 8 is fixed on the photography platform 4.

When a first-stage switching element of a two-stage radiation switch (not shown) is switched on, the control means 80 controls the bias switching means 42 to connect the negative pole of the power supply 40 to the first conductive layer 11 so that a DC voltage is applied between the first conductive layer 11 and each of the elements 15a of the second conductive layer 15 to electrostatically charge the conductive layers 11 and 15. By doing so, a U-shaped electric field is formed between the first conductive layer 11 and each of the elements 15a within the solid-state detector 10 with the element 15a being the bottom of the field.

Thereafter, when the second stage switching element of the two-stage radiation switch is switched on by the imaging staff, radiation is directed toward the breast 8 from the radiation source 2 based on the instruction from the control means 80. When the solid-state detector 10 is exposed to the radiation transmitted through the breast 8, i.e., the recording light carrying radiation image information of the breast 8, positive/negative charge pairs are produced in the recording photoconductive layer 12 of the solid-state detector 10. The negative charges of the pairs are drawn toward each of the elements 15a along the distribution of the electric field described above and stored in the storage section 17 formed at the interface between the recording photoconductive layer 12 and charge transport layer 13. The amount of negative charges stored in the storage section 17, i.e., the charges having the polarity of the latent image is substantially proportional to the amount of radiation transmitted through the subject, so that the electrostatic latent image is now carried by the charges having the polarity of the latent image. Thus, the electrostatic latent image is recorded on the solid-state detector 10. Meanwhile, the positive charges produced in the recording photoconductive layer 12 are drawn to the first conductive layer 11 and re-coupled with the negative charges injected from the high-voltage power supply 40 and disappear.

The control means 80 automatically drives the pressing plate moving means 55 to withdraw the pressing plate 7 from the position to press the breast 8 to the position to release the pressing after the voltage applied to the solid-state detector 10 is terminated.

This is because if the solid-state detector 10 is vibrated while the voltage is applied thereto, noise is added to the electrostatic latent image, and the mode of operation described above may prevent the problem of noise generation.

Thereafter, the conductive layers 11 and 15 are short-circuited through the bias switching means 42, then the reading light source section 20 is activated to provide reading light and the entire surface of the solid-state detector 10 is scanned with the reading light by moving the reading light source section 20 in the longitudinal direction of the elements 15a, i.e., the sub-scanning direction by the moving means 50, and the currents generated by the scanning and flowing through the solid-state detector 10 are detected by the current detecting means 30 based on the instruction from the control means 80.

When the aforementioned scanning is performed, positive/negative charge pairs are produced in the reading photoconductive layer 14, and the positive charges of the pairs move swiftly through the charge transport layer 13 by the attraction of the negative charges (charges having the polarity of the latent image) stored in the storage section 17 tore-couple with the charges of the latent image and disappear at the storage section 17. Meanwhile, the negative charges produced in the reading photoconductive layer 14 are re-coupled with the positive charges injected into the second conductive layer 15 and disappear. In this way, the negative charges stored in the solid-state detector 10 are dissolved by the charge re-coupling, and the movement of the charges in the charge re-coupling produces electric currents in the solid-state detector 10.

These electric currents generated in the solid-state detector 10 are detected in parallel (simultaneously) by each of the charge amplifiers 34a connected to each of the elements 15a respectively. Each of the signals detected by each of the charge amplifiers 34a is sampled and held by each of the sample-and-hold circuits 34b. The voltages, each corresponding to each of the elements 15a, sampled and held by respective sample-and-hold circuits are outputted sequentially from the multiplexer 34c such that they are switched sequentially in the order in which the corresponding group of elements 15a is arranged, which are then outputted sequentially from the multiplexer 33 such that they are switched sequentially in the order in which the elements 15a are arranged. The signals sequentially outputted from the multiplexer 33 are converted to digital signals through the A/D conversion section 32, and stored into the memory 31 as digital image signals.

The amount of current flowing through the solid-state detector 10 generated by the scanning of the solid-state detector 10 with the reading light is proportional to the amount of charges of the latent image or electrostatic latent image, so that the signals obtained by detecting the currents represent the electrostatic latent image, thereby the electrostatic latent image may be read out.

As described above, the present invention may prevent noise arising from the vibrations caused by the movement of the pressing plate and driving of the electric motor used for the moving means during the image signal reading by performing the reading after the pressing plate is withdrawn, thereby the degradation in S/N ratio for the image signals is avoided.

The identical effects may be obtained when the mammographic image obtaining apparatus is configured such that the pressing plate is withdrawn after the image signal reading is completed as shown in FIG. 5B.

In the preferred embodiment of the present invention, a mammographic image obtaining apparatus of automatic releasing type in which the pressing plate is automatically released after imaging has been described as an example. For the apparatus of manual releasing type in which the pressing plate is manually released after imaging, the control means may be adapted to inhibit the movement of the pressing plate during the image signal reading.

Further, in the preferred embodiment of the present invention, an electrostatic recording medium of optical reading system as described, for example, in U.S. Pat. No. 6,268,614 has been described as the solid-state detector, but the present invention is not limited to this, and the identical effects may be obtained if an electrostatic recording medium of TFT reading system is used.

What is claimed is:

1. A mammographic image obtaining system comprising:
a solid-state detector that records image information when exposed to radiation carrying said image information and outputs image signals representing said recorded image information,
a reading means for reading out said image signals outputted from said solid-state detector,
a photography platform having said solid-state detector,
a pressing plate for pressing the breast of a subject onto said photography platform,
a moving means for moving said pressing plate between a position to press said breast of said subject and a position to release said pressing; and
a control means for controlling said moving means,
wherein said control means controls said moving means such that said pressing means is moved by said moving means while said image signal reading is not being performed by said reading means.

2. A mammographic image obtaining system according to claim 1, wherein said solid-state detector comprises a set of layers layered in the order of a first conductive layer; at least one photoconductive layer; and a second conductive layer, and records image information as an electrostatic latent image when exposed to radiation carrying said image information while a predetermined voltage is being applied between said first and second conductive layers and generates electric currents as image signals when scanned with reading light after said first and second conductive layers are short-circuited.

3. A mammographic image obtaining apparatus according to claim 2, wherein said control means controls said moving means such that said pressing means is moved by said moving means while voltage is not being applied between said first and second conductive layers.
